(19) United States
(12) Patent Application Publication
(10) Pub. No.: US 2019/0262275 A1
(43) Pub. Date: Aug. 29, 2019

(54) MULTI-DIMENSIONAL STRUCTURES FROM PEPTOID OLIGOMERS AND METHODS OF MAKING

(71) Applicants: BATTELLE MEMORIAL INSTITUTE, Richland, WA (US); WASHINGTON STATE UNIVERSITY, Pullman, WA (US)

(72) Inventors: Chunlong Chen, Richland, WA (US); Mingming Wang, Richland, WA (US); Teng-Yue Jian, Richland, WA (US); Haibao Jin, Richland, WA (US); Yuehe Lin, Pullman, WA (US); Yang Song, Mill Creek, WA (US); Dan Du, Pullman, WA (US)

(73) Assignees: BATTELLE MEMORIAL INSTITUTE, Richland, WA (US); WASHINGTON STATE UNIVERSITY, Pullman, WA (US)

(21) Appl. No.: 16/406,869

(22) Filed: May 8, 2019

Related U.S. Application Data
(63) Continuation-in-part of application No. 15/880,768, filed on Jan. 26, 2018.
(60) Provisional application No. 62/451,478, filed on Jan. 27, 2017.

(51) Int. Cl. A61K 9/51; B82Y 5/00; B82Y 40/00; B82Y 15/00; C07K 2/00; C07K 1/30; C07K 1/02; A61K 47/42; A61K 47/56

(52) U.S. Cl. CPC A61K 9/5169; B82Y 5/00; B82Y 40/00; B82Y 15/00; A61K 47/56; C07K 1/306; C07K 1/02; A61K 47/42; C07K 2/00

(57) ABSTRACT

Materials and methods are described for forming self-assembled peptoid structures that are extremely stable, crystalline, free-standing and self-repairing are described. Based on the peptoid design, peptoid membranes in a 2D arrangement were able to roll into single-walled nanotubes with tunable sizes, diameters, thicknesses and stiffnesses as well as tailorable functions result. Crystalline nanomaterials made through this facile solution crystallization and anisotropic formation process are highly tailorable and exhibit a number of properties advantageous for applications such as water decontamination, cellular adhesion, imaging, surface coating, biosensing, energy conversion, biocatalysis or other applications.

### PRIORITY

This application claims priority from incorporates by reference in its entirety and is a continuation-in-part of U.S. patent application Ser. No. 15,880,768 filed 26 Jan. 2018 which claims priority from U.S. Provisional Patent application No. 62/451,478 entitled Therapeutic Applications for Two-Dimensional Peptoid Oligomers filed 27 Jan. 2017.

### STATEMENT AS TO RIGHTS TO INVENTIONS MADE UNDER FEDERALLY-SPONSORED RESEARCH AND DEVELOPMENT

This invention was made with Government support under Contract DE-AC0576RL01830 awarded by the U.S. Department of Energy. The Government has certain rights in the invention.

### BACKGROUND OF THE INVENTION

In the field of materials science the use and development two-dimensional (2D) structures such as graphene has received increasing interest in the design of new materials and devices, particularly on the micro and nano scale. In particular, the use of such materials is of interest in applications such as bioanalytical devices, electrochemical devices (such as batteries, fuel cells and supercapacitors), membranes for filtration and separation, surface coatings with chemically defined surfaces and sensors to name a few. However, the widespread use of many known 2D materials is limited because the costs are high and the difficulties in engineering, producing and deploying the materials are significant. Furthermore, many of the attempts that have been made through molecular assembly have resulted in structures and materials that lack the robustness for effectual use or meaningful deployment. This is particularly true when the desired application includes interaction with complex conditions such as a therapeutics or drug delivery devices when harsh conditions such elevated temperatures, pressures, pH's, high salt or other conditions exist.

A desire therefore exists to obtain tailorable robust materials that form easily, have a desired ruggedness and provide simple solutions for meeting designated aims. Examples of such aim include but are not limited to mimicking a biological feature such as a cell membrane that performs molecular separations or for capturing or sensing a target material of interest from an environment or for displaying specific biological activity or for performing a task in a microelectronic environment. The present disclosure provides examples of significant advances in this area.

In particular, in addition to the overall synthesis process described particular examples are provided wherein structures created under the outlined schemes were modified as adapted for a variety of applications including delivery of a targeted molecule for a particular targeted application. Examples of such a configuration include delivery of cancer drugs and gene therapy. While these particular examples are provided, these particular examples are merely illustrative of the various types of materials, solutions and inventions that the methodology and structures described herein make possible.

Additional advantages and novel features of the present invention will be set forth as follows and will be readily apparent from the descriptions and demonstrations set forth herein. Accordingly, the following descriptions of the present invention should be seen as illustrative of the invention and not as limiting in any way.

### SUMMARY

The present disclosure provides new methods to synthesize novel nano-materials from preselected peptoid oligomers using incubation in a liquid solution for a preselected period of time whereby the amorphous oligomers self-assemble to form crystalline 2D and 3D nanomaterials. Depending upon design and process conditions including the design and structure of the peptoid oligomer, the processing conditions and the amount of time left in solution, a variety of structures and shapes including membrane-mimetic two-dimensional (2-D) nanosheets, folded sheets, nanotubes and other structures such as nanocrystalline flower structures can result. In some instances, this self-assembly can take place via crystallization in solution while in other instances the self-assembly takes place on a substrate and forms a coating. The resulting crystalline material can be structured to have atomically flat hydrophobic and hydrophilic surfaces, is effective at cellular adhesion and is capable of self-repair.

In some instances the peptoid oligomers include a preselected item configured to perform a function such as connecting with a preselected target, linking with another item, delivering a drug or other therapeutic item, capturinge a designated material, tracinge the passage of a material within a biological matrix, sensinge the presence of a material or perform another desired activity. In one particular embodiment, a peptoid is utilized that contains a functionalized N-terminus, with a lysine-like group having a CO2 binding affinity for CO2 capture. After placement in solution and incubation for a period of time a self-assembled structure results that has demonstrated extreme stability while also being crystalline, free-standing and self-repairing features. Their related crystallization and anisotropic formation process provide a significant advance in the materials arena.

In one embodiment, these peptoid membranes exhibit a number of properties similar to those associated with cell membranes, including forming thicknesses in the 3.5-5.6 nm range, spontaneous assembly at interfaces, thickness variations in response to changes in Na+ concentrations, and the ability to self-repair. While the selection of the underlying peptoids with alternating hydrophilic and hydrophobic di block structures can be utilized to form lipid like membranes the described nanosheets are superior to lipid bilayers and other membrane-mimetic 2D nanomaterials assembled from lipid analogues because: the materials described are more stable, free-standing, and atomically ordered.

In addition, these structures allow for the attachment of a broad range of functional objects at various locations in the peptoid sequence while leaving the basic membrane structure intact and they serve as a robust platform to incorporate and pattern functional objects through large side-chain diversity and/or co-crystallization approaches. These nanomaterials represent a significant step in the development of biomimetic membranes for applications in water purification, surface coatings, biosensing, energy conversion, water management (filtration, support, absorbance, and retention), biocatalysis drug delivery, or other applications such as antibacterial and antifouling applications among others. While various examples are provided in this example and throughout the application the scope of invention is not limited merely to the specific applications described herein.

The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

FIG. 1) shows the structures of one embodiment of a highly stable and crystalline peptoid nano-sheet nano-membrane with a tunable composition and structure.

FIGS. 2 (a)-2(c) show examples of various membrane forming peptoid sequences that contain functional groups at various positions.

FIGS. 3(a)-3(m) show examples of various functionalized peptoid molecules that include dyes, drugs and receptor targeting ligands.

FIG. 4 shows the formation of a nanotube from a nanosheet structure through the rolling up and closure mechanism described in the present description.

FIGS. 5(a)-5(b) show examples of functionalized nanotube structures containing various preselected functional molecules. Other functional molecules include but are not limited to: drug molecules, inorganic clusters, receptor-targeting ligands.

FIG. 6 shows a scheme for application of functionalized peptoid nanotubes for chemo-photodynamic therapy application.

FIG. 7 (a)-7(i) show various examples of data related to the characterizations of peptoid nanotubes conjugated with IR-780 iodide (IR780) photosensitizer.

FIG. 7(a) shows IR780 conjugated peptoid sequences and the scheme showing the assembly of PepIR into nanotubes.

FIG. 7(b) shows a TEM image of PepIR-20 nanotube assembled from 20% PepIR with 80% Pep.

FIG. 7(c) shows an ex-situ AFM image of PepIR-20.

FIG. 7(d) shows a TEM image of PepIR-40 assembled from 40% PepIR with 60% Pep.

FIG. 7 (e) shows ex-situ AFM image of PepIR-40.

FIG. 7 (f) shows a TEM image of PepIR-100 assembled from 100% PepIR.

FIG. 7 (g) shows ex-situ AFM image of PepIR-100.

FIG. 7 (h) shows the high-resolution TEM image showing the tubes wall thickness in (b).

FIG. 7 (i) shows the XRD spectrum of Pep-20 nanotubes.

FIG. 8 shows a scheme for application of the peptoid-based crystalline nanoflower for gene delivery application.

FIG. 9 shows an example of the self-assembly of fluorinated peptoids into crystalline nanoflowers that exhibit efficient cytosolic delivery

### DETAILED DESCRIPTION OF THE INVENTION

The following description includes examples of various embodiments. It will be clear from this description that the invention described is not limited to these illustrated embodiments but that a variety of modifications and embodiments are also possible, contemplated and enabled by this disclosure. Therefore, the following descriptions should be seen as illustrative and not limiting and including all modifications, alternative constructions, and equivalents falling within the spirit and scope of the invention as defined in the claims.

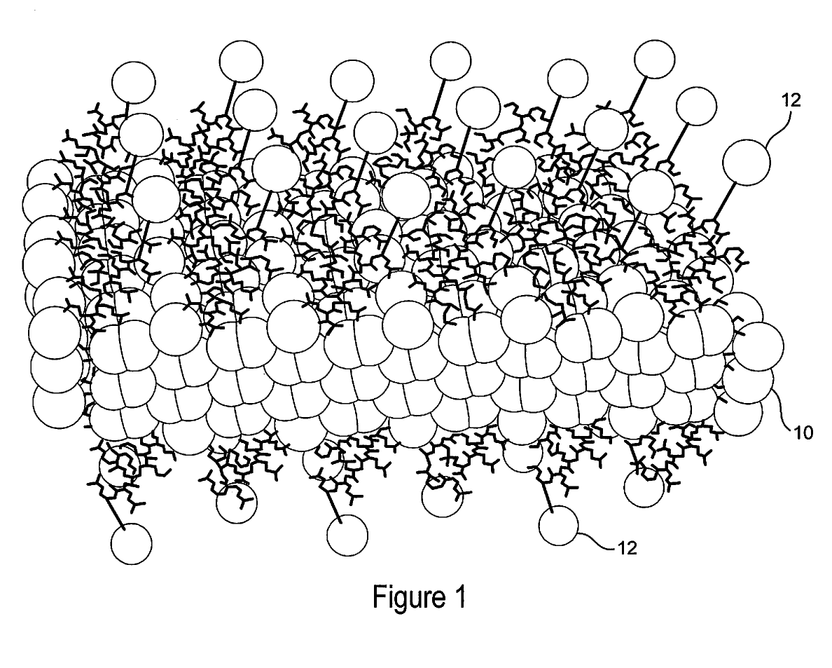

FIGS. 1-5 show various embodiments and descriptions of highly stable, membrane-like two-dimensional sheet structures made from peptoid oligomers that can be variously tuned to create structures including free-standing nanomembranes, coatings, and even highly dynamic and stiff nanotubes. These materials can be configured, functionalized and/or loaded with any of a variety of various target materials so as to facilitate or perform a variety of desired but heretofore very difficult or impossible tasks. These highly-stable and free-standing nanomembranes exhibit self-repair and single-layer coating capabilities, and are formed through a spontaneous self-assembly crystallization process from solution. Their creation does not require interface assisted monolayer compression that attempts in the prior art have required nor are they susceptible to destabilization even when placed in organic solvents.

An exemplary view of one such structure is shown in FIG. 1(a). FIG. 1 shows an assembly of peptoids wherein a portion of the peptoids are assembled to create a nanosheets structure 10 having a series of functionalized molecules 12 extending therefrom. In addition to these elements, additional functionalization or treatments can add other materials of interest or side chains to these structures. Peptoids, or poly-N-substituted glycines, are sequence-defined synthetic molecules that mimic both the structure and function of peptides and proteins, and bridge the gap between biopolymers and synthetic polymers. They are biocompatible and can be easily and cheaply synthesized. In contrast to peptides and proteins, peptoids are highly chemically and thermally stable and offer unique advantages for controlling assembly because lacking backbone hydrogen bonding allows the explicit introduction of interactions through the side chains, thereby leading to functions with high predictability.

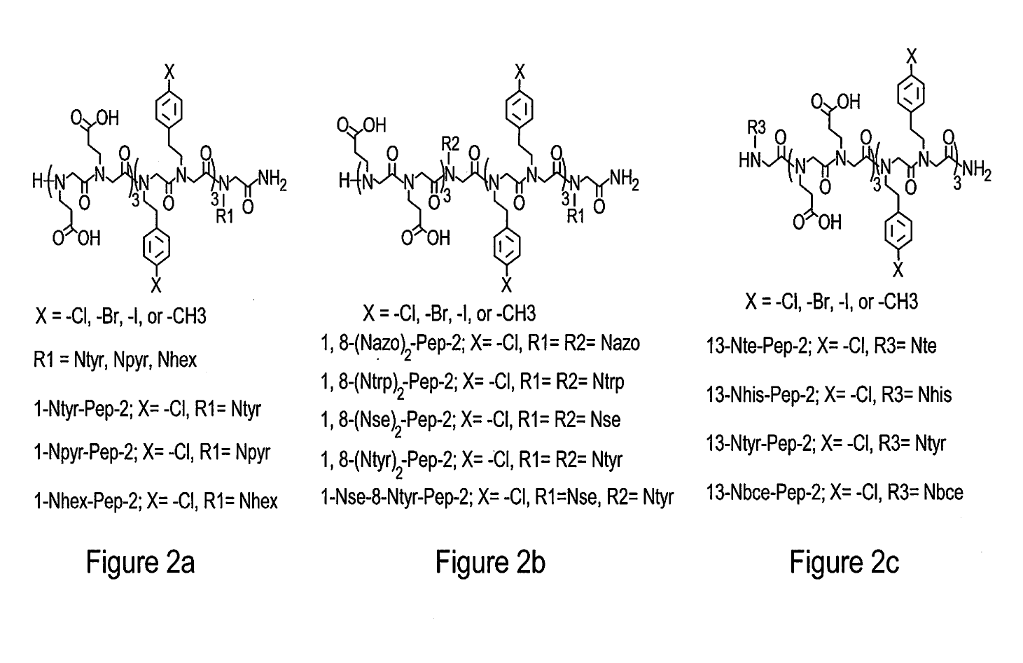

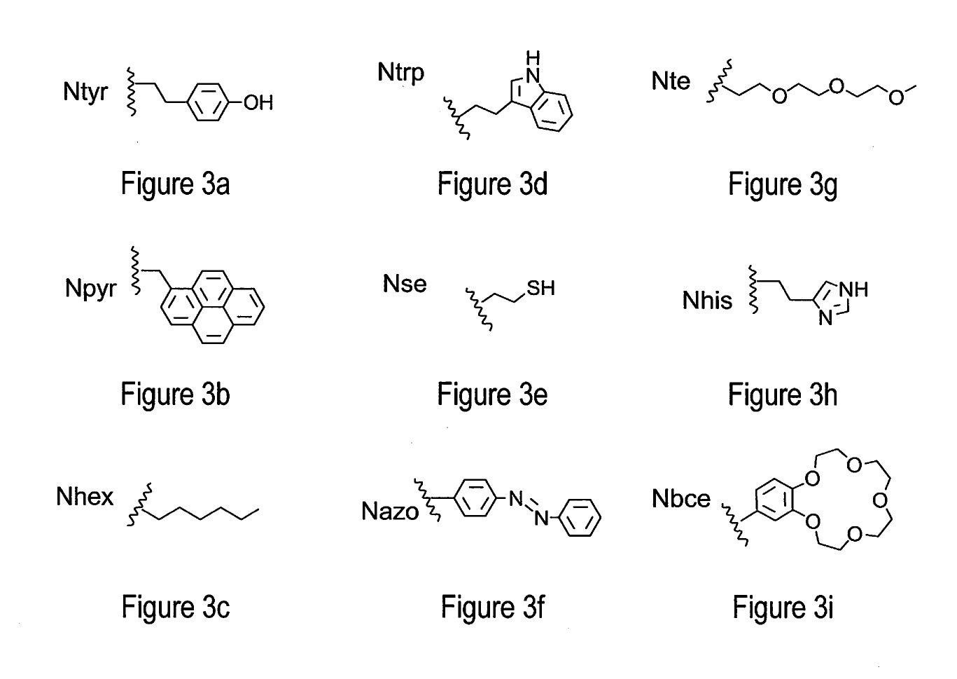

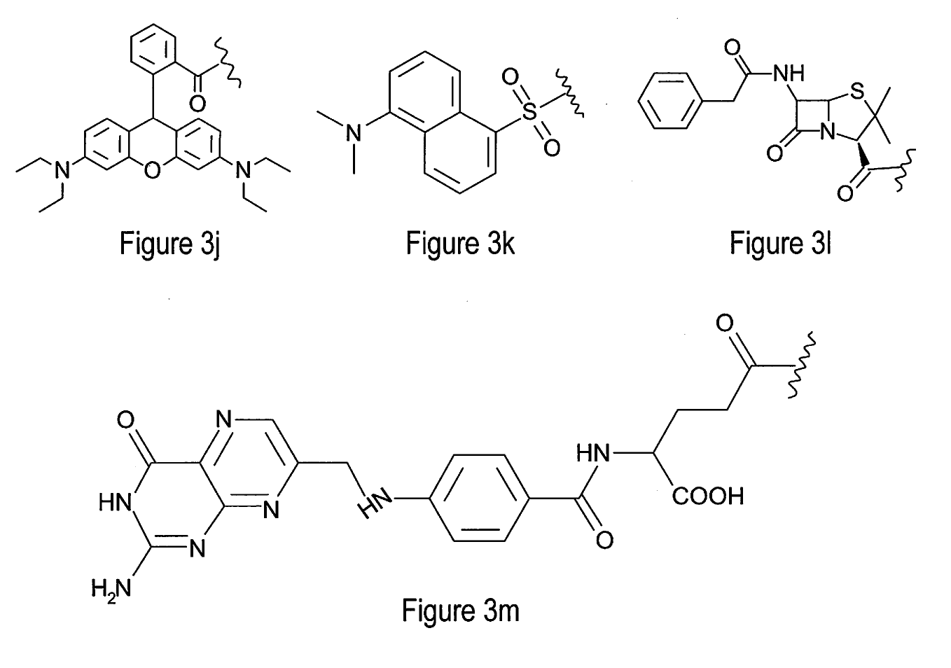

Examples of such molecules are shown in FIGS. 2(a)-2(c)). FIGS. 3(a)-3(m) shows examples of various functionalizations that could take place on these underlying peptoids including the addition of dyes, drugs, and receptor targeting ligands among others. Using these peptoid oligomers as a base, a new class of highly stable and self-repairing 2D peptoid nanomembranes were created using a self-assembly process. The resulting nanomaterials are e free-standing and easy to functionalize. This crystallization method of formation provides the ability to pattern objects into assembled 2D nanomaterials and is significantly different from all previous peptoid 2D materials, which do not demonstrate co-crystallization and are therefore are highly challenged to tolerate the incorporation of bulky functional objects or to create nanoscale-patterning of functional objects within an assembly.

These membrane-mimetic 2D nanomaterial structures can be configured to incorporate and pattern a wide range of functional objects with long-range order to enable collective behaviors (e.g., enhanced structural stability, the ability to self-repair and the high photostability of fluorescent membranes). This represents a long sought after goal in chemistry, materials science, biology and bioengineering.

In one particular application, 12-meric peptoid oligomers capable of self-assembling into two-dimensional sheet structures both in solution and on substrates were created. These 12-mers were synthesized on a synthesizer using a process similar to the one described in U.S. Pat. No. 8,445,632, (The contents of which are incorporated by reference in their entirety) or manually synthesized using a new developed and easy-to-use method set forth below.

In one application manual synthesis of these 12-mer peptoids took place as Rink amide resin (0.09 mmol) was used to generate C-terminal amide peptoids. In the synthesis procedure, the Fmoc groups on the resin were deprotected by adding 2 mL of 20% (v/v) 4-methylpiperidine/N,N-dimethylformamide (DMF), agitating for 40 min, filtering, and washing with DMF. For all DMF washes, 1 mL DMF was added and then agitated for 1 min (repeated five times). An acylation reaction was then performed on the amino resin by the addition of 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF for 5 times. Nucleophilic displacement of the bromine with different primary amines occurred by the addition of 1.5 mL of 0.6 M primary amine monomerin N-methyl-2-pyrrolidone (NMP), followed by the agitation for 10 minutes at room temperature. The monomer solution were filtered from the resin, and washed with DMF for 5 times. The acylation and displacement steps were repeated until the designed peptoid was synthesized.

In other arrangements Pep-3 was synthesized by mixing the resulting rink amide resins (0.09 mmol) containing Pep-2 obtained from automated solid-phase synthesis with a DMF solution of Fmoc-6-aminohexanoic acid (1.5 mL, 0.9 mmol) and 0.50 mL of 50% (v/v) N,N-diisopropylcarbodiimide (DIC)/DMF. The mixture was agitated overnight at room temperature, filtered, and washed well with DMF. The terminal Fmoc group was deprotected by adding 2 mL of 20% (v/v) 4-methylpiperidine/DMF. The mixture was agitated for 40 min, filtered, and washed well with DMF.

CD-APO2 (CD=β-cyclodextrin, APO stands for amphiphilic peptoid oligomers) was synthesized by mixing the resulting rink amide resins (0.09 mmol) containing APO2 obtained from automated solid-phase synthesis with a DMF solution of bromoacetic acid (1.5 mL, 0.9 mmol) and 0.30 mL of 50% (v/v) N,N-diisopropylcarbodiimide (DIC)/DMF. The mixture was agitated for 10 minutes at room temperature, filtered and washed with DMF for 5 times. In the nucleophilic displacement step, 1.5 mL of 0.3 M CD-NH2 in DMF and K2CO3 (100 mg, 0.72 mmol) were added, followed by the agitation for 3 days at 40° C. The monomer solution were filtered from the resin, washed with deionized water for 5 times, and then washed well with DMF.

Peptoids containing N-[4-(2-phenyldiazenyl)phenyl]glycines] (Nazo) were synthesized by mixing 13-Nazo-Pep-2: Rink amide resins (0.09 mmol) containing Pep-2 with 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF. In the nucleophilic displacement step, a NMP solution of 4-Aminoazobenzene (1.5 mL, 0.9 mmol) and tetrabutylammonium iodide (TBAI, 100 mg, 0.27 mmol) was added into the above resins, followed by the agitation for 2 days at 40° C. The resulting resins were first washed with deionized water for 5 times and then washed with DMF for 5 times.

Peptoids containing N-[benzo-15-crown-5-ether]glycines (Nbce) were synthesized by combining 13-Nbce-Pep-2 resins wherein during the nucleophilic displacement step, a NMP solution 4′-Aminobenzo-15-crown 5-Ether (1.5 mL, 0.9 mmol) and tetrabutylammonium iodide (TBAI, 100 mg, 0.27 mmol) were used, followed by the agitation for 2 days at 40° C. The resulting resins were first washed with deionized water for 5 times and then washed with DMF for 5 times.

Peptoids containing [2-(4-imidazolyl)ethylamine]glycines (Nhis) were obtained by mixing 13-Nhis-Pep-2: rink amide resins (0.09 mmol) containing Pep-2 with 1.5 mL of 0.6 M chloroacetic acid in DMF, followed by adding 0.30 mL of 50% (v/v) N,N-diisopropylcarbodiimide (DIC)/DMF. The mixture was agitated for 10 minutes at room temperature, filtered and washed with DMF. In the nucleophilic displacement step, a NMP solution of histamine (1.5 mL, 0.9 mmol) was added into the above resins, followed by the agitation for one hour at 40° C. The resulting resins were washed well with DM.

After introducing Nhis in the peptoid, chloroacetic acid was used instead of bromoacetic acid for all subsequent steps of acylation in order to reduce side product formation as described previously. In the displacement step, primary amines substituted the chloride atom under the condition of agitation 1 hour at 40° C. Peptoids containing N-[2-(1H-indol-3-yl)ethyl]glycine (Ntrp) were formed by mixing 13-Ntrp-Pep-2: Rink amide resins (0.09 mmol) containing Pep-2 with 1.5 mL of 0.6 M chloroacetic acid in DMF, followed by adding 0.30 mL of 50% (v/v) N,N-diisopropylcarbodiimide (DIC)/DMF. The mixture was agitated for 10 minutes at room temperature, filtered and washed well with DMF. In the nucleophilic displacement step, a NMP solution of tryptamine (1.5 mL, 0.9 mmol) was added into the above resins, followed by the agitation for one hour at 40° C. The resulting resins were washed with DMF for 5 times.

After introducing Ntrp in the peptoid, chloroacetic acid was used instead of bromoacetic acid for all subsequent steps of acylation in order to reduce side product formation as described previously. In the displacement step, primary amines substituted the chloride atom under the condition of agitation 1 hour at 40° C. Peptoids containing N-[(1-pyrenemethyl)]glycines (Npyr) were synthesized by mixing 1-Npyr-Pep-2: Rink amide resins (0.09 mmol) containing Pep-2 with 1.5 mL of 0.6 M bromoacetic acid in DMF, followed by adding 0.30 mL of 50% (v/v) N,N-diisopropylcarbodiimide (DIC)/DMF. The mixture was agitated for 10 minutes at room temperature, filtered and washed with DMF for 5 times. In the nucleophilic displacement step, a 3.0 mL methanol solution of 1-Pyrenemethylaminehydrochloride (0.9 mmol) and N,N-Diisopropylethylamine (DIPEA) (0.9 mmol) was added into the above resins, followed by the agitation for 30 minutes at room temperature. The resulting resins were washed with DMF for 5 times.

NHS-Rhodamine-labeled Pep-3 was synthesized by a process wherein, 2 mL of DMF solution of NHS-Rhodamine (0.9 mmol) and 0.50 mL of 50% (v/v) N,N-diisopropylcarbodiimide (DIC)/DMF were added to a solution after the synthesis of Pep-3 and mixed with resins containing Pep-3, followed by the agitation for overnight at room temperature. The monomer solution were filtered from the resin, and washed with DMF for 5 times. The resulting peptoid oligomers resulting from this process have di-block like sequences with hydrophilic or polar side-chains on one end and hydrophobic or apolar side-chains on another end. See FIGS. 2 and 3. When placed into solution under prescribed conditions and allowed to set for a period of time these oligomers self-assembled into 2D nanomaterial membranes generally reflecting the structure shown in FIG. 1 and having desired functionality based upon the configuration of the materials.

Such alternative arrangements provide 2D structures include examples for bioconjugation and/or specific molecular recognition. The evaporation-induced crystallization allows for 12-mer peptoids to be directly used for self-assembly after solid-phase synthesis without purification. In one application, an assembled 2D nanomaterial structure created by this process produced highly stable, sequence-specific and atomically flat connection with both hydrophilic and hydrophobic surfaces (e.g. mica, glass, graphite surfaces). When performed on a substrate, these membranes are capable of covering an entire surface including a well plate.

In other embodiments these materials can act as substrates or scaffolds for tissue engineering, binding specific bio-targets, or act as highly photostable nanoprobes for single particle tracking, as well as in therapeutic, drug delivery or diagnostic applications. For example, covalently bonding a lysine like group on to self-assembling peptoid oligomers allows for 2D peptoid nanosheet structures that have specific CO2 binding affinity have applications as molecular membranes for CO2 separations. Compared to other coating techniques using chemical reagents (e.g. poly-lysine, polyethyleneimine, and collagen) or using plasmonic cleaning, these assembled 2D materials possess a dramatically higher activity and have low cost (Discussed in below) because the multipoint binding can be achieved to specific bio-targets by displaying these 2D sheets with complementary sequences.

In one arrangement, free-standing nanosheet membranes having a ˜4 nm in thickness, up to 5.0 um in width and length in gram scales were self-assembled starting from peptoids that contain a large number of chemically diverse side-chain residues. The use of 12-mer peptoid oligomers in this application, kept the peptoid synthesis cost low (˜$10 per 120 mg of 12-mers; for surface coating: the cost of coating can be as cheap as $0.5 per square meter by using these peptoids). Self-assembly of our membrane-mimetic 2D nanosheets took place not only in solution without requirement of compressing monolayers at water-air or water-oil interfaces, but also on different substrates, thus providing an enhanced and low-cost methodology for atom level coating (coating cost: $0.5 per square meter) and enabling a variety nano and microlevel interactions.

In one particular arrangement this self-assembly of membrane-mimetic 2D nanomaterials from these lipid-like peptoid oligomers took place as lyophilized and HPLC-grade peptoid oligomers were dissolved in a mixture of water and acetonitrile (v/v=1:1) to make 5.0 mM clear solution, this clear solution was then transferred to 4° C. refrigerator for slow evaporation. Suspensions or gel-like materials containing a large amount of crystalline membranes were formed after a few days. Using this arrangement biocompatible and photostable and bio-targeting 2D nanomembranes were assembled from sequence-defined peptoids. The resulting 2D nanomaterials were shown to be capable of regulating the process of membrane receptor internalization, and possess the ability to be rationally tuned for targeted cancer cell imaging and quantitatively tracking their intracellular pathways within live cells using a single-particle tracking technique.

Nanomembranes assembled from these diblock-like peptoids are highly stable and exhibited salt-induced thickness changes. To test the stability of peptoid membranes, they were exposed to a range of solvents as well as high temperature. We demonstrated that peptoid membranes survived when they were placed in a mixture of water and organic solvents, or even in pure organic solvents, such as CH3CN and EtOH, for over 6 hours. They were also stable in 10×PBS buffer (pH 7.4), 1.0 M Tris-HCl buffer (pH 7.4), and 1.5 M NaCl, or after heating to 60° C. in water overnight. Next, we investigated whether these membranes would exhibit salt-induced thickness changes, as is observed with lipid-bilayers. In situ AFM studies showed that when the peptoid membranes were exposed to NaCl solution or PBS buffers of increasing concentration, their thicknesses increased by about 30% from ˜4.2 nm to ˜5.4 nm.

High photostability and quantum yields of peptoid 2D nanomembranes arise from the crystallinity of these nanomaterials. By tuning the surface charge of the 2D nanomembranes using various peptoids such as those with various functional groups, we demonstrated the ability to control the lysosome escape of these materials within live cells during processes. This could assist in a variety of treatments and diagnostics for diseases including drug delivery and other interventions. These 2D nanomaterials can also be used for real-time monitoring of delivery of anticancer drugs with high drug loading rate.

In one specific example, dansyl dye molecules were attached to the membrane-forming peptoids, to create a highly photostable fluorescent nanomembranes that exhibited tunable surface chemistry and tunable surface charging for use in the targeted cancer cell imaging and tunable cellular internalization of pathways within live cells. In another specific example, a series of membrane-forming peptoids were synthesized and modified to by attaching functional groups to the N-terminus of membrane-forming peptoids (Pep-DNS, Pep-FA (FA=folic acid)). These peptoid oligomers were then mixed and co-crystallized using the method described above to form nanomembranes with a tunable density of FA for cancer cell targeting and for tuning the intracellular delivery pathways and kinetics of peptoid membranes. The synthesized highly-photostable nanomembranes can be ultra-sonificated and filtered to form a structure that falls within a narrow size distribution and exhibits tunable surface charges in biological environments. In one specific example, the resulting nanoprobes have a monodisperse nanosheets structure with an average diameter of ˜70 nm.

An important factor for fluorescent probe used in single particle tracking (SPT) is good photostabibity. Peptoid membrane nanoprobes described above kept stable fluorescence properties and structures without aggregation in water even after three months. The high photostability of these peptoid membranes was also confirmed by comparing them with the commercial organic fluorescent dyes Cy5, inorganic quantum dots, and even DNS itself. These results demonstrate that the long-range ordering of surface dye molecules is critical for the synergistic effects of enhanced photostability. This high photostability was also confirmed by experiments of using peptoid membranes as nanoprobes for imaging in the living cells. The nanoprobes showed excellent good in-cell fluorescence stability compared with common cyanine dye (labeling Actin), which allowed us to use it as an endocytic probe in living imaging of cells.

Because peptoid membranes are biocompatible and exhibit high surface area, we expect that these highly stable peptoid membrane nanoprobes offer novel platforms for biological applications. To demonstrate the suitability advantage of nanoprobes as therapeutic agents besides their application as single particle tracking tag, we used nanoprobes as drug carriers to load doxorubicin (DOX)—a commercial drug that widely used in cancer chemotherapy because the aromatic domains of nanoprobes can assist the loading of DOX. The data indicated that there was strong fluorescence quenching between the nanoprobes and DOX. Based on this quenching effect, the DOX-releasing process was in vitro evaluated. We demonstrated that these highly photostable nanomembrane can serve for real-time monitoring of drug release and uptake within living cells due to the time-dependent recovering fluorescence signal of both the nanoprobes and DOX.

The methods and systems described present invention could find application in other areas including molecular separations, electronics, catalysis, optics, energy storage, and biomedicine. Various examples of these membranes, for example those which consist of 3.0-6.0 nm thick bilayers, are of particular interest because they represent a class of 2D materials that have rather unusual properties, such as sequence-specific water and ion transport and the ability to self-repair. Other embodiments of sequence-defined synthetic polymers that mimic lipid amphiphilicity can allow for self-assembly and self-repairing. 2D membrane mimetics exhibit protein-like molecular recognition would revolutionize the development of functional 2D nanomaterials including biomimetic membranes.

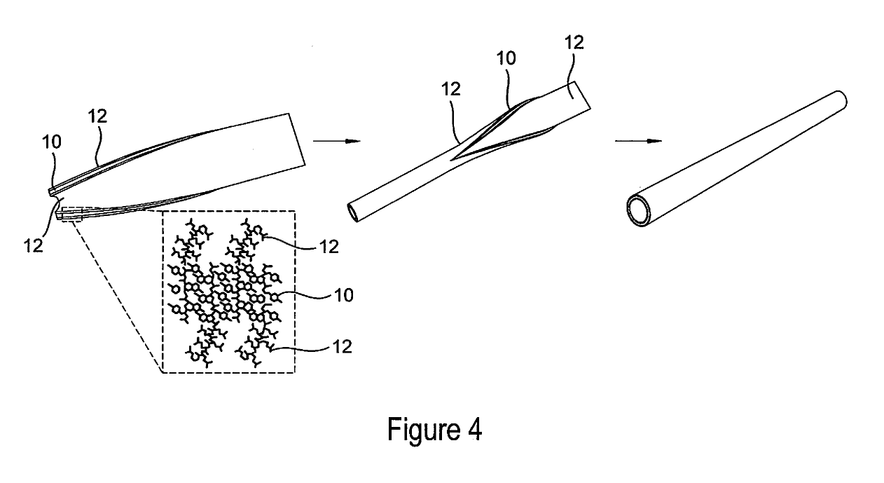

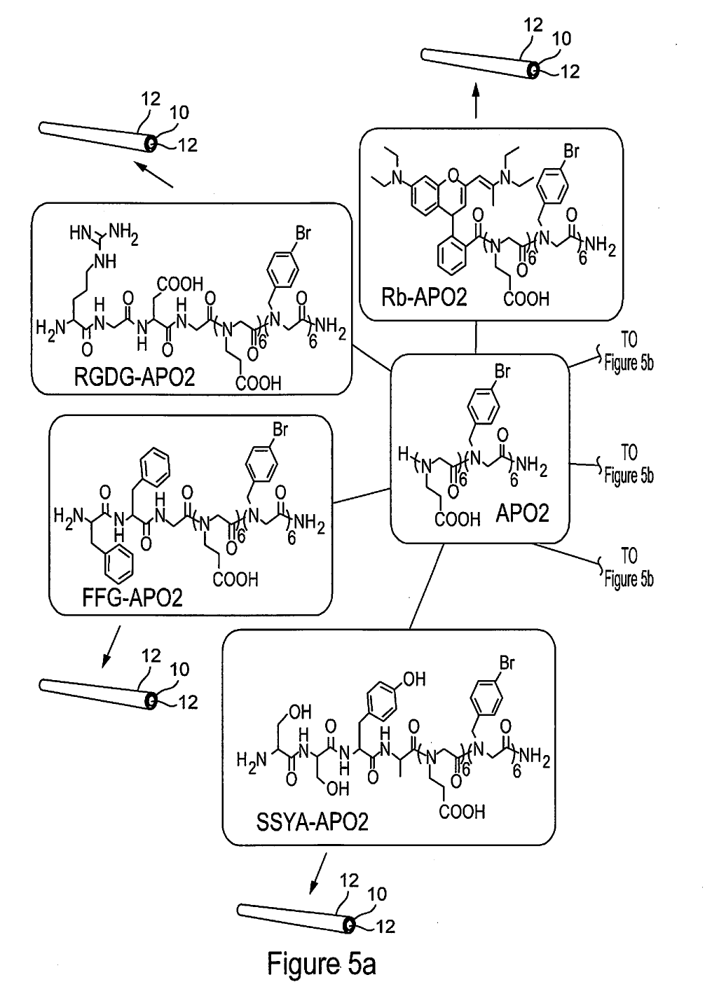

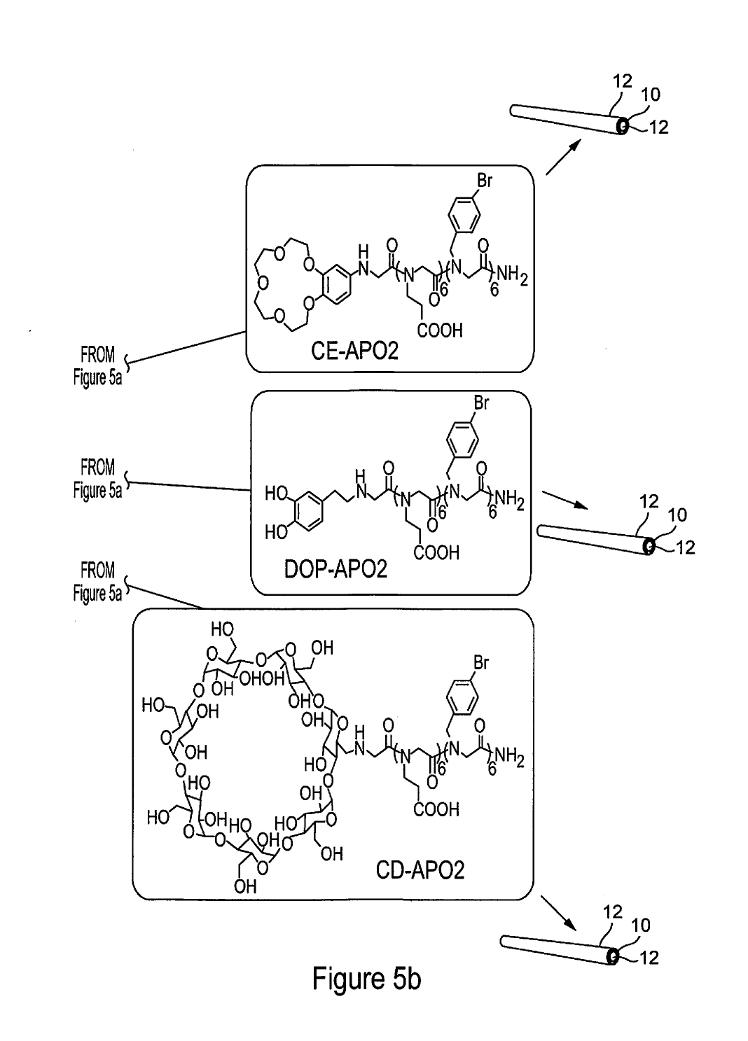

In addition to its arrangement as a tunable membrane, various preselected peptoid oligomers can self-assemble into a variety of shapes and configurations including a designable, stiff and dynamic single walled nanotube. Examples of which are shown in FIGS. 4 and 5. A shown in FIG. 4, formation of these materials takes place ins a process whereby the aforementioned crystalline nanosheet structures assembled from sequence-defined peptoids are rolled, folded and closed to obtain the desired result, single-walled peptoid nanotubes (SW-PNTs).

In one set of experiments peptoid oligomers were formed into PNTs in a process wherein peptoid solutions [5.0 mM, in water and acetonitrile (v/v=50:50, pH 2.5-3)] were left undisturbed at 4° C. for slow crystallization. Gel-like materials containing a large amount of crystalline free-floating PNTs were formed about two or three days later from amorphous phases. Testing on these materials revealed that uniform nanotubes exhibiting a wall thickness of 3.1±0.1 nm, similar to the thickness of bilayer-like peptoid membranes were formed with an average tube diameter of 37.2±2.7 nm. The tube height varied depending upon various conditions suggesting that peptoid nanotubes are dynamic enough to deform. These nanotubes exhibited a length over several micrometers and sonication proved to be an effective way to cut nanotubes in short sections. Interestingly, similar nanotubes formed even when the crystallization solution is at pH 7.4 or pH12. The fact that varying crystallization solution pH did not abolish nanotube formation suggests that hydrophobic interactions contribute significantly to stabilization and formation of nanotubes.

In one investigation, the nanotube formation process was slowed by reducing the concentration of peptoid oligomers to 0.5 mM to capture the nanotube intermediates. In this arrangement APO2 formed uniform nanospheres with a diameter of 26.2±5.1 nm after they were completely dissolved in the mixture of H₂O and CH₃CN. After slowly evaporating the solvent over 30 minutes at 4° C., TEM data showed that peptoids assembled into a mixture of nanospheres with a diameter of 44.9±7.5 nm and later into nanoribbon like sheets with a width of 75-120 nm and length of 200-600 nm. Between 1-72 hours of crystallization, the ribbons began to roll up, fold and close up to form elongated SW-PNTs.

These peptoid nanotubes provide a robust platform for developing biomimetic materials tailored to specific applications. Tuning their surface chemistry and the number of hydrophobic residues of peptoid oligomers allow for variation and tuning of the nanotube wall thickness, diameter and mechanical properties for a particular application. Varying the pH can trigger a reversible contraction-expansion motion of the nanotube. AFM-based mechanical measurements show PNTs can be assembled that are highly stiff (Young's Modulus ˜13-17 GPa).

Incorporation of preselected functional groups within PNTs allows for specialization for various deployments in a variety of applications. To demonstrate this, as shown in FIG. 3, we embedded a variety of chemistries into the structure: a fluorescent dye Rhodamine B (Rb-APO2), crown ether (CE-APO2), biomolecule dopamine (DOP-APO2), peptides sequences (FFG-APO2, SSYA-APO2, and RGDG-APO2), or cyclic host molecule β-cyclodextrin (CD-APO2) at the N-terminus of APO2. These items were made present as peptoid side chains, and the PNT assembly was sufficiently robust to tolerate the addition of functional groups, building functional PNTs with tunable compositions and functions. This ability provides a basis for the creation of a variety of types of structures suitable for performing a variety of tasks including but not limited to water decontamination, drug delivery, therapeutic, diagnostic and other nano and microscale applications.

In one example, such tunable PNTs were used for water decontamination. Dyes containing an azo chemical group (azo-dyes) have been widely used as textile colorants and now become one of the major toxic pollutants in water. Among various strategies that have been developed to remove azo-dyes, physical adsorption is considered to be superior to others due to its high efficiency, ease of operation and low cost. CD-APO2-PNTs were used here as adsorbents for the removal of azo-dyes from water, in which aromatic 4-aminoazobenzene was used as a model azo dye molecule. These CD-APO2-PNTs removed the majority of 4-aminoazobenzene molecules from water within one hour. We believe that this due at least in part to the combination of the function of β-cyclodextrin which is known to encapsulate azo-dyes, especially aromatics, through specific host-guest interactions and a nanotubular structure offers large surface area and high porosity.

In another set of experiments these PNTs were developed for promoting the cell adhesion. Specifically, the glass slide coated with RGDG-APO2-PNTs (FIG. 3) exhibited the most significant adhesion of A549 cancer cells in contrast to the control slides. Such promoted cell adhesion induced by RGD-containing PNTs was also observed during the uptake of sonication-cut-PNTs within A549 live cells.

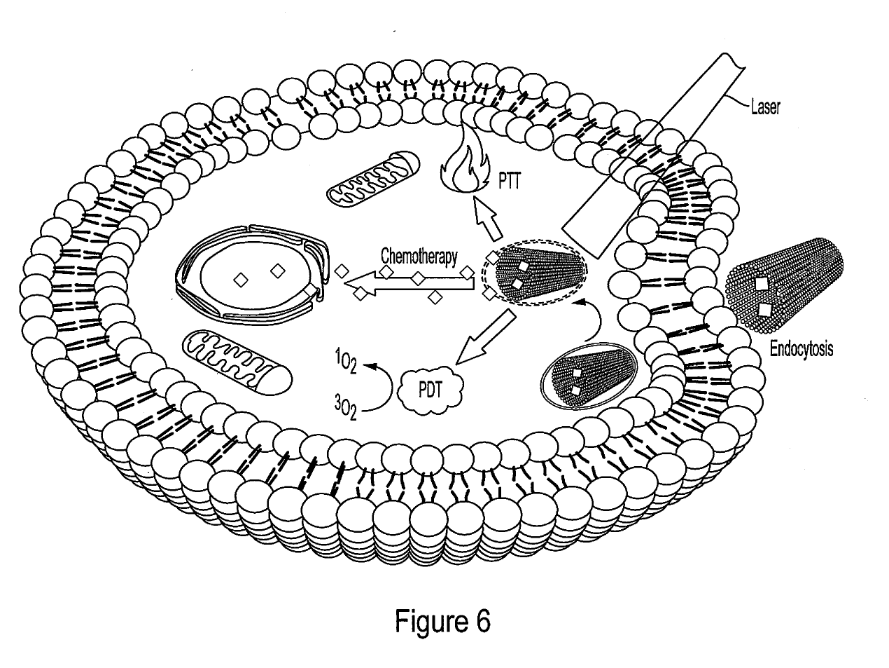

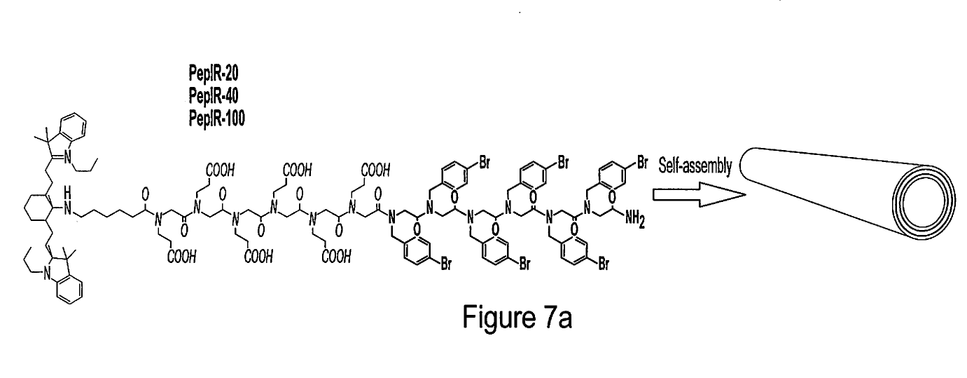

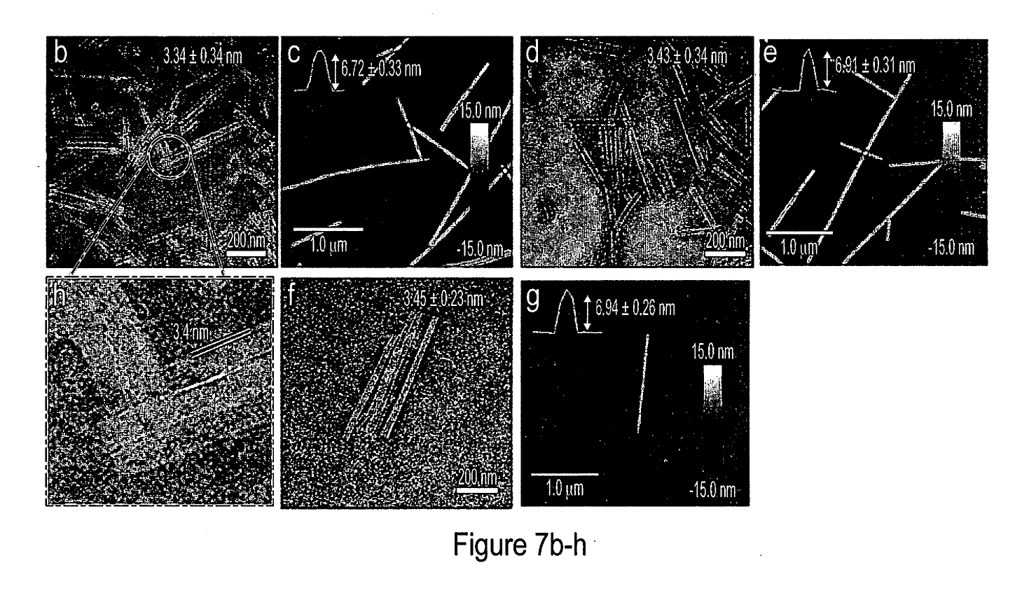

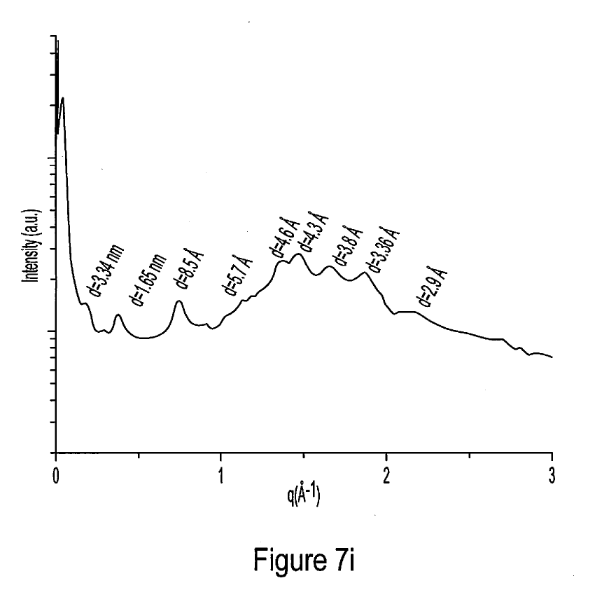

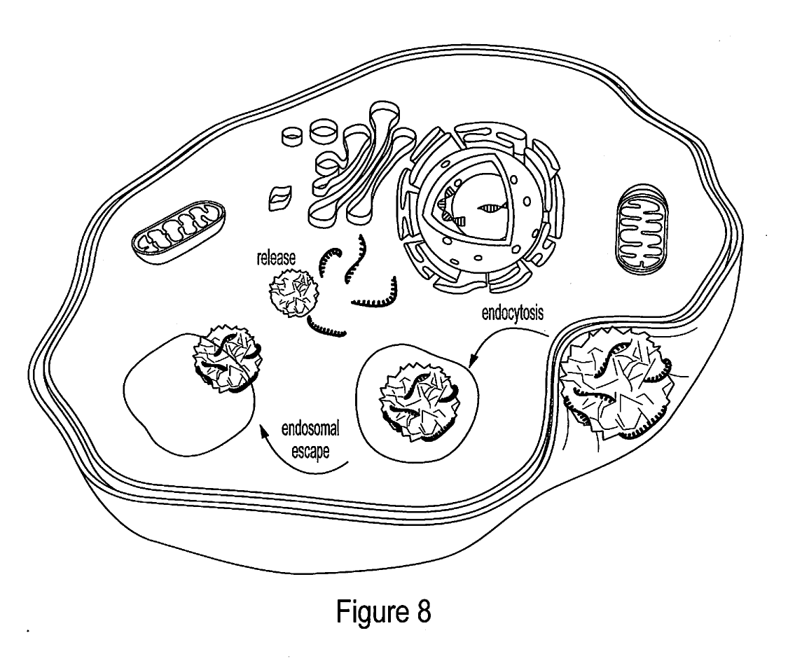

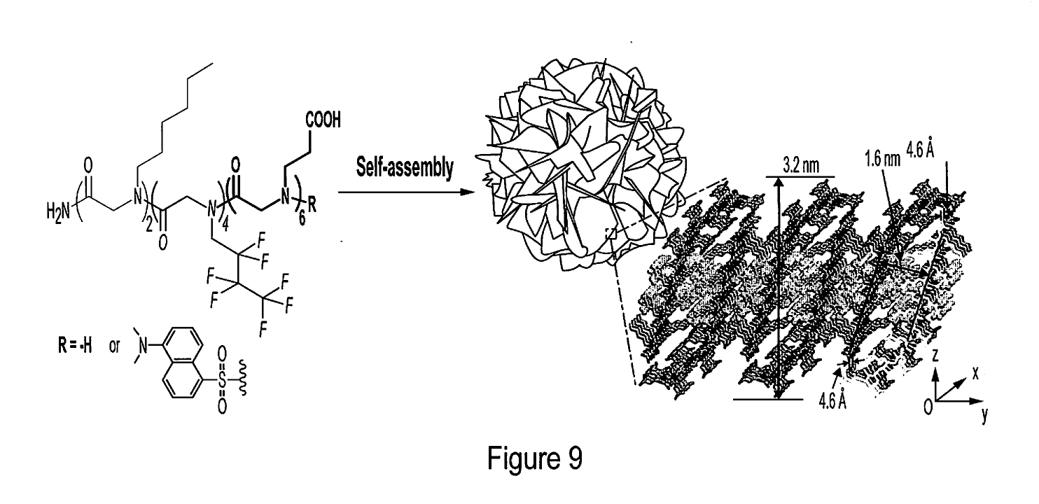

The stability of these peptoid nanotubes was tested as we exposed them to a range of solvents as well as high temperature. Peptoid nanotubes survived when dispersed in alkaline solution (pH=11.94) for over 6 hours. The high stability of these peptoid nanotubes was further demonstrated as they remained intact after being incubated in a mixture of water and CH3CN, or in 1×PBS buffer, or in 1.0M NaCl, or after heating to 60° C. in aqueous solution for 3 hours. Peptoid nanotubes also survived when they were placed in pure organic solvents (e.g. CH₃CN and EtOH) for over 3 hours. In another set of experiments we developed a new family of functional nanotubes assembled from conjugated peptoids. Examples of functional nanotubes are shown in FIGS. 6 and 7 (a)-(i). We demonstrated their applications in water decontamination and cellular adhesion and uptake. The easy synthesis and large side-chain diversity of peptoids enabled us to introduce a wide range of functional groups, such as fluorescent dye, macrocyclic compound, biomolecule, or peptides within peptoid nanotubes. Because peptoids are sequence-defined, highly stable, biocompatible and exhibit protein-like selectivity for molecular recognition, we expect this new type of nanotubes can provide a robust platform for development of biomimetic materials tailored to biological applications, such as chemo-phototherapy (CPT) strategy.

Malignant glioma is one of the most aggressive tumors in the brain and a major cause of death. It exhibits invasive growth into surrounding normal brain tissue. Chemotherapy remains the main treatment but the single therapy result is unsatisfactory due to their inability to address the highly invasive nature of glioma. Recently, phototherapy showed great potential for malignant glioma therapy by using a laser to selectively causing tumor cell apoptosis. For the above reasons, herein, we developed a simple and effective multimodal therapeutic strategy against gliomas by CPT strategy.

One challenge in CPT therapy is the lack of effective, stable and biocompatible carrier which can load drug and near-infrared agents simultaneously. Here, highly stable and crystalline nanotubes were used as the scaffold to precisely display IR780 and load chemotherapeutic drug DOX. Owing to the precise adjustment of IR780 intermolecular distance as a result of high crystallinity of nanotubes, these nanotubes assembled from IR780-conjugated peptoids (PepIR) exhibited a simple, safe and effective platform for simultaneous PDT/PTT, in which DOX were loaded within PepIR nanotubes for synergistic treatment. The combined CPT strategy (FIG. 6) resulted in significantly higher therapeutic efficiency than individual phototherapy or chemotherapy.

Using the previously described solid-phase submonomer synthesis method,³¹ a series of tube-forming peptoids with and without IR780 were designed and synthesized. As shown in FIG. 7a, these peptoid sequences have a polar domain containing six N-(2-carboxyethyl)glycine (Nce) groups and a hydrophobic region having six N-[(4-bromophenyl)methyl] glycine (Nbrpm) groups, and IR780 dye were conjugated at the N-terminus adjacent to the polar domain. Nanotubes with tunable density of IR780 molecules were prepared by co-assembling PepIR with Pep in a variable ratio. As shown in FIG. 7(a), nanotubes assembled from PepIR, 40% PepIR with 60% Pep, or 20% PepIR with 80% Pep all had similar morphologies and structures. (Characterization showed an approximate diameter of 32-36 nm; a wall thickness of approximately 3-3.5 nm, (however materials formed under dryer conditions tended to have a greater wall thickness (nearly twice as thick) and a height between 6.4 and 7.2 nms. X-ray diffraction (XRD) data showed more details of the nanostructure indicating that the nanotubes are highly crystalline and that the spacing in the spectra corresponds to openings of sufficient size so as to allow the placement of dye molecules within the crystalline structures.

The spacing of 5.7 Å is attributed to the ordered packing of aromatic side chains within the hydrophobic Nbrpm₆ segments. The 4.6 Å spacing corresponds to the alignment of lipid-like peptoid chains. The peak at 3.36 Å indicated the spacing between residues along the chain direction. And the presence of extensive π-stacking is evidenced by the peaks at 4.3 Å, 3.8 Å, and 2.9 Å. As the model we proposed in previous work, in the tubular walls, the hydrophobic Nbrpm groups segments are packed with each other and embedded in the center of the tubular wall, while the hydrophilic Nce segments were distributed on the exterior surface of the wall, exhibiting a similar packing as we described in the formation of bilayer-like peptoid nanotubes. These results showed that IR780 dye molecules could be precisely displayed within highly crystalline peptoid nanotubes with a tunable density while remaining the similar framework structure.

The main challenge in the design of dye-doped nanoparticles for phototherapy is to solve the problem of self-quenching of the dyes. Indeed, as flat aromatic structures, PSs tend to π-stack into poorly fluorescent aggregates (H-aggregates), leading to reduced phototherapy efficiency. Highly crystalline nanotubes exhibit well-defined orientations and distances of functional groups, which is an obvious advantage for engineering NIR dye molecules for phototherapy. To demonstrate the use of these PepIR nanotubes for phototherapy, we analyzed their fluorescence spectra. As shown in FIG. 2a, the fluorescence spectra of PepIR nanotubes gradually increased as the assembly percentage of PepIR increased from 20% to 40%. However, when 100% PepIR was used to assemble nanotubes, the resulting nanotubes exhibited a decreased fluorescence intensity which is probably due to the aggregation of IR780 at high concentrations. These results indicated that nanotubes assembled from 40% PepIR with 60% Pep (PepIR-40) are the best candidate for phototherapy among these three types of nanotubes. Besides, the UV-vis absorption of PepIR-40 nanotube in water was significantly higher than those free IR780 dye in methanol or water, which further indicated that the high crystallinity of peptoid nanotubes can restrict the conformational flexibility of grafted IR780 molecules and weaken their potential π-π stacking interaction, thus subsequently reducing their self-quenching effect. These results prove that PepIR-40 nanotubes can effectively prevent the IR780 dye from aggregating in aqueous solution and are good candidates for phototherapy applications.

In another set of experiments PepIR-40 nanotubes with size of ˜130-160 nm were prepared by ultrasonication. TEM image showed that these PepIR-40 nanotubes remained tube morphologies and structures with wall thickness of ˜3.4 nm, a measured diameter of these nanotubes between about 33 and 36 nm, which is consistent with those of pre-sonicated PepIR-40 nanotubes, indicating sonication is an effective way to cut peptoid nanotubes into short length without changing other parameters. After preparation and characterization, the photothermal behavior of the nanotubes was studied by placing the tubes in an aqueous solution and subjecting the tubes to laser irradiation. In these experiments, the temperature of the PepIR-40 aqueous solution increased to 49.2° C. in 5 min under the 808 nm laser irradiation, and over the maximum temperature was 50.1° C. These temperatures were high enough to cause significant hyperthermia damage to cancer cells. The results proved the efficient photothermal conversion ability of PepIR-40.

In addition, to verify the photodynamic effects, the ¹O₂ generation of PepIR-40 was also evaluated using 9,10-anthracenediyl-bis(methylene) dimalonic acid (ABDA) as an indicator. ABDA could undergo oxidation to yield an endoperoxide in the presence of a singlet oxygen, resulting in the decreased absorption of ABDA. In the absence of PepIR-40, there was no obvious decrease in ABDA absorption after exposure to 808 nm laser irradiation. However, the absorption peak of ABDA gradually decreased with 808 nm laser irradiation after PepIR-40 was added to the solution supporting the notion that ¹O₂ was generated and PepIR-40 can be used in PDT.

In another instance DOX, a well-known anticancer drug, was chosen as a model drug to be loaded in this PepIR nanotubes (PepIR-DOX) with loading content up to 26.4%, The UV-vis spectra of the PepIR nanoparticles showed the characteristic peak of IR780 at ˜800 nm. After drug loading, the characteristic peak of DOX was also observed in the spectra, illustrating that DOX had been successfully loaded into the PepIR vector. DOX loading was further confirmed by the fluorescence spectra of PepIR, free DOX and PepIR-DOX. The DOX-loading had not negative effect upon the intracellular phototoxicity of PepIR.

Cells treated with PepIR and exposed to 808 nm laser irradiation showed strong green fluorescence, suggesting there was some ROS generation in the U87MG cells and further demonstrating the potential of PepIR for PDT therapy. When the ¹O₂ scavenger ascorbic acid (AA) was premixed with PepIR before irradiation, the green fluorescence signal became weak, which proved that the green fluorescence was observed only in the presence of ¹O₂. In addition, a burst of ROS could disrupt the normal physiological redox state, resulting in the destruction of mitochondrial membrane potential (MMP), as indicated by using rhodamine 123 (Rho 123). Compared with the nonradiative group, bright green fluorescence was observed in the U87MG cells after incubation for 4 h with PepIR was followed by irradiation, indicating the destruction of mitochondria. These results proved the ROS-generating ability of PepIR under 808 nm laser irradiation.

In addition to these individual adaptations, the synergistic effects of PepIR-DOX on the growth of the U87MG cells were investigated by the MTT assay. PepIR under 808 nm laser irradiation or loaded with DOX exhibited increased cytotoxicity in a concentration-dependent manner after 48 h incubation. For the PepIR-DOX nanoparticles under 808 nm laser irradiation, over 70% of the cells were killed at a very low peptoid tube concentration, indicating that this combined treatment had significantly high therapeutic efficiency against glioma cell growth. Moreover, the half-maximal inhibitory concentration (IC₅₀) of each treatment and the combination index (CI) was calculated to be 0.323 (<1), confirming a fairly high synergistic therapeutic effect.

In another set of experiments photosensitizer (PS)-doped peptoid crystalline nanotubes, in which IR780 was attached at the N-terminus of tube-forming peptoid sequences, were co-assembled and developed as an efficient platform to deliver DOX for the simultaneous chemo-PDT/PTT trimodal treatment of glioma. In this versatile system, the IR780 dyes were effectively packed and separated from each other by peptoid arms, the self-quenching of the donors was inhibited, and the dyes remained stable in water and showed excellent ROS production and photothermal conversion ability in the experiments. Moreover, efficient DOX-loading was achieved due to the large surface area of the nanotubes, contributing to an efficient CPT strategy against the glioma cells. Enhanced antitumor efficiency was confirmed by the CLSM and MTT assays. Given the above results and the unique properties of peptoids and peptoid nanotubes, the synthesized multimodal DOX-containing PepIR nanotube-based system in this work offers great promises for future glioma therapy in clinical applications.

In another set of experiments 3D crystalline structures referred to as nanoflowers 30 were formed with fluorinated peptoid oligomers. These structures provided a variety of advantages including the ability to deliver various markers and treatment agents intracellularly to assist in the treatment of a variety of conditions. An example of such an embodiment is provided hereafter.

Crystalline nanoflower-like particles were designed and synthesized from fluorinated sequence-defined peptoids. The crystallinity and fluorination of these particles enabled highly efficient (nearly 80%) cytosolic delivery with minimal cytotoxicity. These particles, for example, can carry mRNA for gene transfection, demonstrating the generality of the nanocrystals. This avoids various problems which exist with other methods wherein nanocarriers are usually taken up by endocytosis and are prone to entrapment within subcellular compartments, such as endosomes or lysosomes which prevents the interaction of the macromolecule drugs (e.g., nucleic acids, peptides and proteins) with target molecules and potential degradation in these compartments.

In this example, dansyl (DNS) labeled crystalline nanoparticles fluorinated peptoid crystals (FPC) were formed using the self-assembly process that was described earlier. Negatively stained TEM images of these arrangements showed that the FPC particles exhibit an interesting flower-like morphology. A close look at this transmission electron microscopy (TEM) data showed that these flower-like FPC particles are made of layers of sheets. Atomic force microscopy (AFM) image showed that the FPC nanoparticles display uniform size distribution with a dia meter of ≈150 nm. Dynamic light scattering measurements showed that these FPCs exhibited a narrow size distribution with an average diameter of ≈130 nm and negatively charged surfaces (−50 mV) in phosphate-buffered saline buffer. XRD (x-ray diffraction) data showed that FPC particles are highly crystalline showing the bilayer-like packing of amphiphilic peptoids. The alignment of peptoid chains, which leads to a spacing of 4.6 Å between peptoids along x-direction. A 1.6 nm spacing corresponds to the distance between two peptoid backbones in the direction of fluorinated groups facing each other along y-direction. Both hydrophobic and fluorine-fluorine interactions of these fluorinated peptoid nonpolar domains imparted high stability to the FPC. A similar nonfluorinated peptoid Nhex6Nce6 (Nhex=N-(6-hexyl) glycine; Nce=N-(2-carboxyethyl)glycine), which the four fluorinated side chains were replaced with four Nhex groups. As we expected, the formation of crystalline particles was not observed

As an application of their use, FPC particles were incubated with H1299 cancer cells at three different concentrations (10, 50, and 100×10-9 m). Testing revealed that the fluorescence intensity of the cancer cells increased when the FPC concentration rose from 10 to 100×10-9 m, confirming that FPC uptake is concentration dependent. At a concentration of 100×10-9 m, FPCs were efficiently taken up by cultured H1299 cancer cells. Further testing demonstrated that although a minority of the FPCs were immobilized at the plasma membrane (appearing in both epifluorescence and TIRFM imagings), most of the FPCs were found beyond the plasma membrane within the cytosol. Images from whole cells further showed that the FPCs were efficiently delivered into the cell and that most internalized FPCs were motile and excluded from the nucleus.

In addition, uptake rates under different conditions were examined. The resulting data showed that the cellular uptake of the FPC was almost completely stalled at temperatures of 4° C. or lower, obstensibly because the endocytosis of the FPC was energy dependent. The addition of various materials was demonstrated to have varying effects upon the uptake of FPC materials in the cells. The addition of cytochalasin D, for example, led to a decrease in the uptake of the FPCs. In contrast, the cellular uptake rates of the FPCs were only slightly affected by the presence of methyl-b-cyclodextrin (inhibitor of caveolae-mediated uptake), nocodazole (tubulin depolymerizing agent), and chlorpromazine (inhibitor of clathrin-mediated uptake). These results revealed that the endocytosis of the FPC mainly occurs through the macropinocytosis pathway.

With the successful cellular internalization of the FPC via endocytosis, additional investigation on the endo/lysosomal escape characteristics of FPC were performed. The delivery of the FPC from the endo/lysosomes into the cytosol was analyzed using two independent methods. First, the diffusive motion of the FPC in the cytosol was tracked and characterized. The cytoplasm is a highly crowded environment with a densely packed network of organelles, macromolecules, and cytoskeletal elements. Generally, the cytosolic motion of molecules exhibits Brownian motion, which shows a large diffusion coefficient. Such movement will be random and will have a short range. On the other hand, endosomes associated with molecular motors can show a long range and have directed motility. Hence, if the NPs escape from endosomal compounds, it is unlikely that the particles can move a long distance in the cytoplasm. After the cells were treated with FPCs, we observed two types of motility behaviors of the FPCs in the cytosol: short-range hop movements and long-range directional motility. The experiments showed that ≈80% of the FPCs underwent the first type of movement. The diffusion coefficient of these FPCs was ≈1.5-fold higher than that reported for endosomes suggesting that the vast majority of the FPCs escaped from the endosomes and were delivered to the cytosol.

Colocalization studies of the FPCs within the endosomal compartments revealed that most FPCs were indeed not located in endosomes and instead were accumulated in the cytosol. The few FPCs that did colocalize with dextran were mostly immobile most likely attributable to the trapping of FPCs in immobile endosomal compartments. Further testing and review of FPCs with early endosomes (EEs), late endosomes (LEs) and lysosomes revealed that with 1 hour of incubation, most of the FPCs still colocalized with EEs in the whole plasma region (52±8%; N=12 cells). However, after increasing the incubation time to 2 h, we found low colocalization with both EEs (21±6%; N=12 cells) and LEs (20±5%; N=12 cells), confirming that the FPCs indeed escaped from the endosomal components. The analysis showed that the number of FPCs trapped in EEs gradually decreased, while the colocalization degree of the FPCs with LEs remained at a constant low level for the whole observation period, indicating that the FPCs did not interact with LEs and stayed in the cytosol after escaping.

To investigate whether the FPCs were taken up through membranes by creating transient holes, we investigated whether the internalization process of FPCs was accompanied by the escape of specific organelle tracking dyes. First, H1299 cells were coincubated with FPCs (no DNS label) and calcein (calcein can remain in endosomes or lysosomes unless it is released to the cytosol after the endosome membrane is disrupted). The cells that were incubated with FPCs and calcein exhibited a solely punctate endosomal distribution of calcein, indicating that the FPCs did not facilitate the uptake of calcein into the cytosol.

We also investigated whether the FPC could cause the escape of cytosol tracer molecules during uptake. For this purpose, H1299 cancer cells were incubated with calcein-AM. Testing showed that the internalization process for FPCs did not create any leakage of calcein-AM from the cells. In addition, the cytotoxicity of the FPCs was evaluated by using the standard 3-(4,5-dimethylthiazol-2-yl)-2,5-diphenyltetrazolium bromide (MTT) assay with H1299 cells. Almost 91% cell viability was observed even with the incubation of H1299 cells with FPCs at the maximal concentration of 25×10-6 m for 24 h. This demonstrates the low cytotoxicity of the FPC and reflects that FPC was taken up through cell membranes and escaped from endosomes while causing minimal cytotoxicity and no overt membrane pore formation.

This is believed to result from the order surface groups organization on the FPC, which results in unique properties due to the close surface apposition of hydrophilic or hydrophobic groups. Hence the FPC will exhibit excellent solubility and low protein-binding avidity, which induces nondisruptive fusion of FPC with fluid mixed layer (such as cell membrane) and subsequent penetration through the cell membrane and endosome membrane without creating pores and inducing cytotoxicity. An additional feature of such an arrangement may be the adsorption of ssDNA on FPCs attributable to the synergistic physisorption of nucleobases on the surface of peptoid nanostructures. This interaction of ssDNA with FPC was strong and unaffected by bovine serum albumin (BSA) or 10% fetal bovine serum (FBS). In contrast, the double-stranded complexes (ssDNA hybridized with its target) can result in continuous release of the target away from the surface of the FPCs. Functionalization of the FPCs with ssDNA did not affect their efficient cytosolic delivery. Time-lapse imaging analysis revealed that cytosolic ssDNA fluorescence could be recorded upon the exposure of cells to the FPC/ssDNA complex and that the ssDNA fluorescence increased after long-term incubation, thus demonstrating the very efficient detachment and release of the FPCs. The ssDNA fluorescence was evenly distributed throughout the cytosol of the whole cell, further demonstrating that FPC-mediated ssDNA delivery allows the ssDNA to enter the cytosol and bypass endo/lysosomes. Further testing demonstrated that FPC effectively formed complexes with mRNA following increasing FPC concentrations while the adsorption of mRNA by FPC protected mRNA against nuclease degradation, a prerequisite for intracellular delivery of mRNA.

With successful binding mRNA with FPC, we focused the investigation on the gene-delivery process by studying the transfection efficacy of mRNA-enhanced green fluorescent protein (EGFP) in H1299 cancer cells mediated by FPC. A constant amount of mRNA-EGFP was pretreated with different concentrations of FPC and then incubated with H1299 cancer cells for 48 h. When H1299 cells were transfected with FPC/mRNA complex, the transfection efficacy can reach as high as 71% at a FPC concentration of 20×10-6 m, which is higher than the transfection rate using Lipofectamine MessengerMAX for mRNA delivery in H1299 cells (67%). The excellent mRNA transfection performance mediated by FPC/mRNA complex can also be demonstrated in other cells. Compared to cells treated with Lipofectamine/mRNA complex, similar EGFP fluorescence signals were observed due to comparable transfection performance provided by FPC. However, the Lipofectamine has been flawed for their incapability of gene molecule protection upon nuclease degradation, resulting in quick inactivation under serum-rich conditions.

Adsorption of mRNA by FPC also protected the mRNA against nuclease degradation, a prerequisite for mRNA transfection. Lipofectamine/mRNA complex was treated with RNase and then applied to gene transfection in H1299 cancer cells. The As shown in FIG. 5D, the transfection efficacy of Lipofectamine decreases dramatically from 67% to 19% after RNase treatment. In contrast, significant EGFP fluorescence signals in the FPC/mRNA complex-treated cells can still be detected after treatment of RNase, indicating strong enzymatic cleavage protection of FPC on mRNA. Results from all of the testing showed that supported fluorination played an important role for FPC to hold good performances on intracellular trafficking and gene delivery.

The present disclosure provides a variety of examples of new materials and methods for their synthesis. These materials and their associated methods in their various permutations allow for the specialization and tailoring of micro and nano structured devices that are useful in a variety of applications including but not limited to targeted material capture, for example as a part of environmental clean-up, or industrial processing or mining, or sensing or detection, in biological applications such as discovery, or diagnostics or therapeutic applications, in applications such as drug delivery, molecular sensing, biological imaging or biomimetic materials tailored to specific applications or application in nanoelectronics.

While various preferred embodiments of the invention are shown and described, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A method to synthesize a material comprising the step of:
placing preselected peptoid oligomers having a preselected functionalized complimentary sequence configured to connect with a preselected target, in a liquid solution for a preselected period of time whereby said oligomers self-assemble to form a crystalline structure configured to perform a preselected function

2. The method of claim 1 wherein the preselected function is delivering a drug.

3. The method of claim 1 wherein the preselected function is capturing a designated material.

4. The method of claim 1 wherein the preselected function is tracing the passage of a material within a biological matrix.

5. The method of claim 1 wherein the preselected function is sensing the presence of a material.

6. The method of claim 1 wherein the preselected function is delivering a therapeutic material.

7. The method of claim 1 wherein the crystalline structure is a nanotube.

8. The method of claim 1 wherein the crystalline structure is a nanoflower.

9. A two dimensional nanomembrane-like material comprising:
modified peptoid oligomers self-assembled in a crystalline structure.

10. The material of claim 9 crystalline material has atomically flat hydrophobic and hydrophilic surfaces.

11. The material of claim 9 wherein the peptoid oligomers include a preselected complementary sequence configured to connect with a preselected target.

12. The material of claim 11 wherein the crystalline structure further comprises a preselected material configured to perform a preselected function.

13. The material of claim 11 wherein the preselected function is delivering a drug.

14. The material claim 11 wherein the preselected function is capturing a designated material.

15. The material of claim 11 wherein the preselected function is tracing the passage of a material within a biological matrix.

16. The material of claim 11 wherein the preselected function is sensing the presence of a material.

17. The material of claim 11 wherein the peptoid is functionalized at the N-terminus to include a lysine-like group having a CO2 binding affinity.

18. A three-dimensional crystalline therapeutic agent comprising a material having three-dimensional fluorinated peptoids arranged in a nanoflower shape.

19. The three dimensional crystalline therapeutic agent of claim 18 wherein the fluorinated peptoids have the following structure: [chemical structure]
